(19) United States
(12) Patent Application Publication
(10) Pub. No.: US 2023/0034978 A1
(43) Pub. Date: Feb. 2, 2023

(54) PROCESS FOR MEASURING EFFICACY OF PAIN THERAPY

(71) Applicant: Mikhail Nemenov, Mountain View, CA (US)

(72) Inventor: Mikhail Nemenov, Mountain View, CA (US)

(21) Appl. No.: 17/827,629

(22) Filed: May 27, 2022

Related U.S. Application Data
(60) Provisional application No. 63/193,655, filed on May 27, 2021.

(51) Int. Cl. A61B 5/00

(52) U.S. Cl. CPC A61B 5/4041; A61B 5/0082; A61B 5/4848; A61B 2562/0238

(57) ABSTRACT

A process for utilizing a diode laser to illuminate at least one A-delta nerve fiber and at least one C nerve fiber to produce a mechanistic biomarker for measuring efficacy of a pain therapy.

### CROSS REFERENCE TO RELATED APPLICATIONS

Applicant claims the benefit of Provisional Patent Applicant Ser. No. 63/193,655 filed May 27, 2021, all of which is incorporated by reference herein.

### FIELD OF THE INVENTION

This invention relates to medical processes for using diode lasers for evaluating pain therapy.

### BACKGROUND OF THE INVENTION

Applicant's U.S. Pat. No. 7,402,167 dated Jul. 22, 2008 contains an excellent summary of the state the art of evaluating pain therapy as of the filing date of that patent. The discussion in that patent is also an excellent discussion as of the filing of above cited provisional application. That background is hereby incorporated by reference into this application as well as the rest of that patent. That background includes discussions of chronic pain prior art research dealing with methods and devices for simulating pain, Ion channel research and the use of lasers in pain research. It also includes an excellent summary of A-Delta nerve fibers and C nerve fibers and specifically, spontaneously active C mechano-insensitive fibers. That background section also includes a summary of Applicant's prior research as of the filing date of the application for that patent. That background section represents a good discussion of the background for the present invention although some discussions of more recent developments are referred in the Detailed Description of the Present Invention.

In previous U.S. Pat. Nos. 7,402,167B2 and 8,029,553B2 the device and method for selective activation of C or A-delta nerve fibers in humans and animals were described. In patent application US20190008440 it was also described that diode laser selective C fiber stimulation can induce axon reflex flare could be used for measurement of neuropathic spontaneous pain and C-mechano-insensitive nerve fibers. The evidences of these properties were approved in the following clinical and preclinical pain research:

1-10 (see reference list)

What is needed is a process for utilizing a diode laser to illuminate at least one A delta nerve fiber and at least one C nerve fiber to produce a mechanistic biomarker for measuring efficacy of a pain therapy.

### SUMMARY OF THE INVENTION

The present invention provides a process for utilizing a diode laser to illuminate at least one A-delta nerve fiber ant at least one C nerve fiber to produce a mechanistic biomarker for measuring efficacy of a pain therapy. The process is useful for evaluating a pain therapy. The diode laser is used to illuminate the A delta nerve fibers and the C nerve fiber with a plurality of laser beams to produce a set of intensity responses. The intensity responses are measured to create a set of ratios of the intensity responses prior to applying the pain therapy and a second set of ratios are obtained after the therapy has been applied. Both sets of ratios are compared to determine the efficacy of the pain therapy. The patient could be animal or human. In preferred embodiments, the diode laser is adapted to produce laser beams at a desired wavelength between 600 nm and 2000 nm. Wavelengths of 980 nm are preferred. The invention is especially important when the pain is chronic pain. Preferred beam diameters are between 0.5 mm and 2 mm for illuminating the at least one A delta nerve and between 5 mm and 10 mm for illuminating the at least one C nerve fiber. Preferred laser beams power is between 10 W and 30 W for illuminating the at least one A delta nerve and a power of between 0.6 W and 6 W for illuminating the at least one C nerve fiber. A preferred measured intensity response is an intensity of painful or painless sensations. The measured intensity response could be a first recognition of painful or painless sensation or an intensity of power or laser pumping current to produce a first recognition of painful or painless sensation or to produce a specified level of pain. The measured intensity response could be an intensity of pain or an intensity of power to produce a specified level of pain.

### BRIEF DESCRIPTION OF THE DRAWINGS

FIG. 1 shows a cross section of patient skin that Illustrates nerve fiber denervation (decreased nerve fiber density in pain patients and ability of diode laser (DLss) to access deep located C mechano-insensitive (CMi) fibers.

FIG. 2A shows the flare area 18 second after DLss stimulation of CMi fibers receptive field, the size of flare over 30 mm, the diameter of diode laser beam that activate CMi fibers is below 5 mm.

FIG. 2B shows a time dependent blood perfusion flare response.

FIG. 3A shows a boxplot comparison of ratio of healthy patients (Con) and neuropathy populations; chemotherapy induced polyneuropathy without pain (CIPN) or with pain (pCIPN), painful diabetic neuropathy (pDPN). Mean, SEM and range are shown, p**<0.01, p*<0.05.

FIG. 3B shows a scatter plot of C pain and Aδ detection thresholds. Most painless CIPN patients segregate to upper left quadrant. CIPN patients with severe pain tended to aggregate in upper right, while those in upper left had low pain report.

FIG. 3C shows a ROC curve (receiver operating characteristic curve is a graphical plot that illustrates the diagnostic ability of a binary classifier system as its discrimination threshold is varied) shows that DLss C:Aδ ratio provides greater diagnostic power to distinguish painful and painless groups (AUC 0.84) when compared to QST measures; heat pain threshold (HTP) AUC 0.55, warm detection threshold (WDT) AUC 0.58.

### DETAILED DESCRIPTION OF THE PREFERRED EMBODIMENTS

The present invention provides a first fully translatable mechanistic biomarker of NEOROPATHIC PAIN that could be used as end point measure of efficacy of pain therapy (pharmacological or device based), and that is in FDA classification of biomarkers named RESPONSE biomarker. Besides, it could be used as DIAGNOSTIC biomarker and for selection of personalized pain therapy.

OPN—ongoing neuropathic pain

p-OPN—peripheral ongoing neuropathic pain

PN—peripheral neuropathy

DLss—diode laser fiber selective stimulation (C or Ad fibers)

IENFD—intraepidermal nerve fiber density (morphological examine/test of peripheral neuropathy)

CMi—C mechano-insensitive fibers (fibers responsible for mediation of neuropathic pain),

UENS Utah Early Neuropathy Scale (patient neurological examine)

VAS visual analogue scale to measure pain (0 no pain 10 non tolerable pain)

C:Aδ ratio of diode laser intensities that cause threshold detection of pain sensations

EndpointS and other Tools (BEST) FDA-NIH initiative that defined description of biomarkers

QST quantitative sensory testing are psychometric measures of perception

DBRCT double blind research clinical

WDT and CDT are QST measures: warm and cold detection thresholds

HPT and CPT are QST measures: heat and cold pain thresholds

MPT is QST measure mechanical pain threshold

CIPN chemotherapy induced peripheral neuropathy

DPN diabetic peripheral neuropathy

pCIPN painful CIPN

pDPN painful DPN

CNS Central nerve system

fMRI functional magnetic resonance imaging (fMRI) is a technique for measuring and mapping brain activity that is noninvasive and safe.

EEG electroencephalography is an electrophysiological monitoring method to record electrical activity of the brain

Now we suggest an improvement of the previous patents and patent application we suggest that ratio of diode laser intensity that evokes C fiber activation at first recognition of painful (Cpain) or painless (Cdet) sensation divided to laser intensity that evokes A-delta fiber activation at first recognition of painful (Adelta-pain) or painless (Adelta-det) sensation is marker of pain that may separate patients with painful and painless neuropathy and measure efficacy of pain therapy regardless patient age, previous treatment and severity of neuropathy. The diode laser intensity could be measured in output power (mW) of laser pumping current (mA). Besides, the diode laser intensity that induces axon reflex flare and activation of C mechan-insensitive fibers as it described in patent application US20190008440 may substitute in C/Adelta ratio Cdet. The C pain/Adelta detection ratio is preferable compared to Cpain/Adelta-pain, Cdet/Adelta-det and Cdet/Adelta ratios.

This improvement is critical for estimation efficacy of pain therapy, because group analyses is needed. Spontaneous activity of C nerve fibers preferably C mechano-insensitive fiber mediated to central nerve system is responsible for pain. Ability of pain treatment to knock out spontaneous activity of pain nerve fibers (nociceptors) and its propagation to Central Nerve. The diode laser stimulation accesses the spontaneously active pain mediated fibers. However, measurement of pain and detection threshold are also affected by nerve fiber density in skin. Nerve fiber density (skin innervation) is individual for each pain patient and changing with age, treatment and severity of neuropathy. Such difference is critically affected an ability to compare the pain patients. Therefore, C thresholds has to be normalized to account nerve fiber density. Adelta thresholds are very sensitive measure of fiber density and accounting of Adelta fibers thresholds (pain or detection) in form of C/Adelta ratio is normalized C thresholds regardless of age and severity of neuropathy. Therefore, C/Adelta ratio allows to separate patients with painful and painless neuropathy and to measure efficacy of pain therapy.

Examples of applications of the present invention include:

1. Application of the above ratio in patients with diabetic and chemotherapy induced neuropathy for separation painful and painless neuropathy regardless patient age and neuropathy severity

2. Application ratio for developing pain therapy on example of lidocaine as end point biomarker

These examples are discussed in detail below.

### Purpose of the Invention

The purpose of this invention is acceleration of novel pain therapy development by biomarker signature of peripheral ongoing neuropathic pain (ONP). Spontaneous peripheral ONP is a frequent presenting symptom, and often the most debilitating feature of peripheral sensory neuropathy, severely impacting quality of life. Painful peripheral neuropathy (PN), whether idiopathic, or associated with chemotherapy (chemotherapy induced polyneuropathy) and other drugs, diabetes, metabolic syndrome or hereditary disease afflicts 6-10% of the US population. There is a lack of effective treatment leading to opioid prescription in most PN patients, with resulting morbidity. Development of medications that reduce spontaneous peripheral nociceptor transmission is hindered by lack of a practical Response biomarker specific for these fibers.

This novel peripheral ONP biomarker signature based on assessment of C and Aδ nociceptors using a diode laser to selectively stimulate these fibers (DLss). Spontaneous activity of cutaneous C fibers, mainly C mechano-insensitive fibers (CMi), are responsible for mediation of peripheral ONP. DLss allows assessment of spontaneously active CMi fiber-mediated neuropathic pain across cutaneous depth, while assessment of Aδ fibers provides a surrogate measure of PN distal axonal loss.

Applicant proposes that DLss measures as a novel BEST defined Response biomarker based on its specificity for peripheral origin of neuropathic pain. The optimized DLss measures is significantly correlate with neuropathic pain intensity in peripheral ONP patients.

An example of clinical trials for measurement pain therapy efficacy is based on lidocaine patch. An example of separation patients with painful and painless neuropathy is presented for patients with painful and painless diabetic and chemotherapy groups of patients.

This invention will deliver the first practical mechanistic biomarker signature of Response to peripherally active neuropathic pain medications, ready for advanced prospective clinical validation studies. DLss C:Aδ ratio is distinguished between placebo and active treatment arms, and significantly correlate with extent of pain reduction. When combined with patient reported change in neuropathic pain into a hybrid primary endpoint, C:Aδ ratio ameliorate placebo effect and improve statistical power in early Phase pharmaceutical trials. Therefore, this invention will facilitate the development of effective, peripherally acting neuropathic pain treatments and may provide the first neuropathic pain biomarker for FDA certification.

The purpose of this invention is to provide a novel biomarker signature of peripheral ongoing neuropathic pain (p-ONP) for efficacy of peripherally acting pain therapy. Spontaneous p-ONP is a frequent presenting symptom, and often the most debilitating feature of peripheral sensory neuropathy, severely impacting quality of life. Painful peripheral neuropathy (PN), whether idiopathic, or associated with chemotherapy induced polyneuropathy (CIPN) and other drugs, diabetes, metabolic syndrome or hereditary disease afflicts 6-10% of the US population(2). There is a lack of effective treatment leading to opioid prescription in most PN patients, with resulting morbidity(3). Development of medications that reduce spontaneous peripheral nociceptor transmission is hindered by lack of a practical Response biomarker specific for these fibers(4-6).

Applicant has preliminary evidence for a novel p-ONP biomarker signature based on assessment of C and Aδ nociceptors using a diode laser to selectively stimulate these fibers (DLss)(7) (8) (9) (10). Spontaneous activity of cutaneous C fibers, mainly C mechano-insensitive fibers (CMi), are responsible for mediation of p-ONP(11) (12). C-DLss allows assessment of spontaneously active CMi fiber-mediated neuropathic pain across the cutaneous depth, while assessment of Aδ fibers provides a surrogate measure of PN distal axonal loss(10). Our preliminary data demonstrates that C:Aδ ratio is reliable when repeated, and significantly distinguishes between groups with painless and painful chemotherapy induced and diabetic PN. By contrast, other available measures of neuropathy severity (e.g intraepidermal nerve fiber density (IENFD)) or small fiber function (e.g. quantitative sensory testing) do not(13, 14). Cutaneous axon flare specifically reflects CMi activation (15). We show that C-DLss induces neurogenic flare and therefore may act as a valuable additional biomarker component (US20190008440).

DLss C:Aδ ratio as a novel BEST defined Response biomarker(1) based on its specificity for peripheral origin of neuropathic pain. The optimized DLss C:Aδ ratio is significantly correlate with neuropathic pain intensity in p-ONP patients independent of PN etiology and severity. Further, change in C:Aδ ratio is significantly correlate with reported pain response to treatment with a peripherally acting neuropathic agent, and distinguish between placebo and drug arms.

Application of ratio in patients with diabetic and chemotherapy induced neuropathy for correlation of severity of painful neuropathy and DLss C/Adelta ratio regardless patient age and neuropathy severity

DLss was correlated to measures of pain and neuropathy severity in 50 participants, age 30-80 with length dependent sensory predominant peripheral neuropathy (idiopathic, chemotherapy induced, prediabetic and diabetic). Each participant was exam with Utah Early Neuropathy Scale (UENS), and IENFD to establish duration and severity of neuropathy and pain disability. DLss, quantitative sensory testing, and axon-flare testing was assess function of sensory afferents. Mean of twice daily app-based visual analogue scale (VAS) over a week will serve as the neuropathic pain comparator. Axonal flare response, age, and neuropathy severity was explored as inputs of C:Aδ ratio biomarker correlation to reported neuropathic pain.

Outcome:
1. The ratio C pain/Adelta det was significantly different between groups of patients VAS below 2 and higher than 4, p<0.01, regardless of patient age or IENFD.
2. The flare intensity/area was significantly different between groups of patients VAS below 2 and higher than 4, p<0.01, regardless of patient age or IENFD.

70 p-ONP participants to with mean pain VAS >40 mm who undergo baseline DLss, and pain measures. Response to VAS was compared to change in C:Aδ ratio following treatment with peripherally active lidocaine patch (5%), or placebo patch using a 7 day treatment cross-over design with 14 day washout between treatment arms.

Outcome: Change in C:Aδ ratio was distinguish lidocaine from placebo treatment groups, p<0.01

### Impact Statement

This invention delivers the first practical mechanistic biomarker signature of Response to peripherally active neuropathic pain medications, ready for advanced prospective clinical validation studies. DLss C:Aδ ratio will distinguish between placebo and active treatment arms, and will significantly correlate with extent of pain reduction. When combined with patient reported change in neuropathic pain into a hybrid primary endpoint, C:Aδ ratio may ameliorate placebo effect and improve statistical power in early phase pharmaceutical trials. This tool will facilitate the development of effective, peripherally acting neuropathic pain treatments and may provide the first neuropathic pain biomarker for FDA certification.

### Significance

Peripheral ongoing neuropathic pain (p-ONP) is common, debilitating, expensive, and has no satisfactory treatment. P-ONP occurs in the setting of peripheral neuropathy (PN) associated with diabetes16, 17 and metabolic syndrome18, HIV and other viral infections19,29, as a side effect of drugs including chemotherapy 21, 22, with genetically defined syndromes23 and as an idiopathic condition 24-26 p-ONP has a similar peripheral origin across all these peripheral neuropathies, and affects 7-10% of people worldwide 27, 15.7% in the US 2. This prevalence is increasing as the population ages and cancer survival28, obesity, dyslipidemia and diabetes become more common 29. Pain is often the most disabling aspect of PN30-32, and has been estimated to cost $25 billion annually in direct medical costs,(33) and $3.6 billion in lost work33-34. Only a minority of P-ONP patients find relief in currently available neuropathic pain agents.(35) More than 65% of PN patients are prescribed opioids 36, leading to side effects and physical dependency37, 38. Development of effective pharmaceuticals directed at ONP is hampered by a lack of quantitative and mechanistic biomarkers of treatment response that can supplement and validate patient self-report 39-41. The development of such a pain response biomarker has been identified as a critical unmet need by the Congressionally mandated NIH Help End Addiction Long Team (HEAL) Initiative. This invention provides a robust BEST-defined Response mechanistic biomarker focused on ONP's peripheral origin.

### Comparison to Alternative Biomarker Candidates

Neither brain-based functional measures of pain, nor currently available measures of peripheral small fiber function are strong candidate biomarkers of peripheral ONP. Development of p-ONP requires both peripheral sensitization and central reinforcement 42, 43. Biomarkers for central pain perception intensity have been developed using fMRI 44, EEG alpha wave characteristics 44, 45, and field mapping of transcranial magnetic stimulation46. These central measures hold promise for predicting risk for transition to chronic pain, but because of the integrative physiology of CNS pain perception, are not specific as surrogates for peripherally evoked neuropathic pain 4-6, 45. Validation of biomarkers specific to peripheral transmission is of great import because this transmission plays a critical role in chronic ONP development and maintenance. Agents that block peripheral pain transmission have been shown to produce essentially complete relief of spontaneous ONP in PN patients12. Validation of a biomarker specific to this transmission step will facilitate clinical testing of similar agents.

Morphological measures of small diameter peripheral axons such as intraepidermal nerve fiber density (IENFD) after punch skin biopsy or corneal confocal microscopy are sensitive surrogates of severity of spinothalamic afferent loss 47, but do not correlate well with intensity of neuropathic pain48-51. Similarly, quantitative sensory testing (QST) are psychometric measures of perception to a particular sensory modality that are neither specific to the neuroaxis site of injury (i.e. PN or multiple sclerosis yield similar loss of vibration or cold discrimination), nor correlated with pain severity. Peripheral needle microneurography allows enumeration of spontaneously active peripheral fibers 52, 53, but is tedious, time consuming, painful and qualitative.

Anatomy and physiology of cutaneous nociceptive fibers underlies the specificity of DLss for spontaneously evoked neuropathic pain.

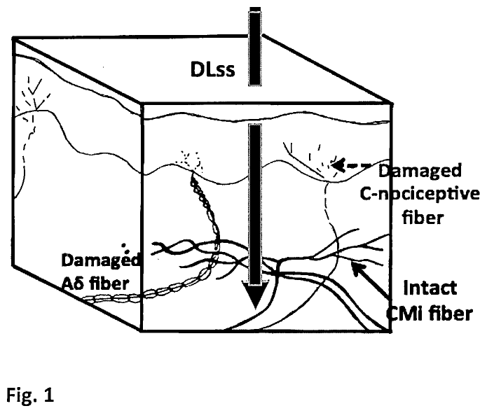

Two types of nerve fibers found within the epidermal and dermal skin layers, lightly myelinated Aδ and small unmyelinated C fibers, transmit nociceptive (pain) information 54. Spontaneous activity of small fibers is associated with painful peripheral neuropathy (PN) in animals and humans 55-57. A specific subtype of C fibers, the C mechano-insensitive (CMi) fibers, are abnormally spontaneously active in patients with painful PN 56, 58. CMi fibers are located primarily in the dermis, (FIG. 1), often spared early in humans and animal models of PN 59, 60, have widely branching afferent arbors, are relatively insensitive to mechanical stimuli, but respond to noxious heat and chemicals61. When activated, these fibers release chemokines that cause vasodilation and sensitize surrounding fibers. This suggests CMi are primarily responsible for the evoked neuropathic pain of PN. DLss, for the first time, allows selective sampling of these fibers.

In contrast, Aδ fibers and other C fibers that penetrate from the dermal layer into the epidermis are primarily responsible for mediating cutaneous sensations of sharp/pin, cold or heat, but are less likely to generate neuropathic pain by spontaneous activation11, 54. In experimental models, ablation of these epidermal nociceptive fibers leads to loss of pain sensitivity, rather than pain 54, 62-64. Because PN is often rigidly length dependent, the distal axons of these very longest epidermal nerve fibers are often lost first, resulting in reduced sensation for pin/sharp or cold/heat 65. Thus, nerve fiber loss alone is neither necessary nor sufficient to peripheral development of neuropathic pain13, 14, 51. We anticipate that DLss C:Aδ ratio will correlate significantly with ongoing pain perception, but not with age and neuropathy severity.

DLss noninvasively, and selectively stimulates cutaneous small fiber populations, including dermal mechano-insensitive C fibers responsible for neuropathic pain. DLss works by introducing energy to skin in a way that activates cutaneous nociceptive fibers until depolarization of axonal tips or modality-specific receptors evokes conscious sensation. The power (Amperage) of induced laser energy necessary to evoke the patient awareness of pain represents the output of DLss testing. Other techniques also introduce energy to cutaneous nerves including surface heat to generate a heat pain threshold 66, and laser evoked potentials after focal nerve stimulation with CO2 laser 67, 68. The energy from these techniques are distributed very superficially in skin. As a result they do not activate CMi fibers unless very high levels of energy are used, which risks burns or skin injury 69. By contrast, DLss, 980 nm wavelength energy penetrates skin evenly to a depth of more than 1 mm. Modulation of DLss stimulus area size and duration selectively stimulates abnormally sensitized cutaneous fibers regardless of their depth: superficially (0-0.1 mm) or deep in skin (0.3-1 mm), evoking sensory threshold or pain sensation either from shallow Aδ fibers or deeper CMi fibers. The ratio of amperage necessary to evoke threshold pain sensation at these two depths, the C:Aδ ratio is the innovative surrogate measure of spontaneous peripheral neuropathic pain, which this project will deliver as a Response biomarker.

Scientific premise and biological specificity is robust for DLss as a biomarker for spontaneous CMi activation. In extensive work with both human volunteers and animal models, one of the PIs, Nemenov, carefully defined the biological properties and axonal targets of DLss, developed appropriate protocols for its use, and compared DLss to similar techniques to stimulate cutaneous axons.
Focused 980 nm diode laser light with duration below 200 ms selectively activated heat-sensitive Aδ fibers, experienced as prickling pain. Wider stimulus with duration over 1 sec activates heat sensitive CMi fibers but does not activate Aδ fibers, resulting in delayed and long-lasting pain7, 8.
Diode laser infrared light penetrates deeply and uniformly heats the epidermis and dermal layers, when compared to contact heat or Tm:YAG and CO2 lasers 70-72. Compared to these techniques, DLss heat is homogeneous across epidermal and dermal layers, resulting in nearly identical activation thresholds within the same class of nociceptive fibers regardless of depth 72. Because DLss radiation is absorbed in skin water rather than melatonin, it is not dependent on skin pigmentation, and works across species 8, 62, 73.
Because the diode laser does not contact the skin, wavelength is stable and intensity is directly defined by diode current, DLss provides greater consistency of heating in repeated measures compared to other techniques 74. DLss induced temperature variability less than 3% in repeated trials.
In diabetic PN, C and Aδ fibers respond differently to DLss. Impairment of nociception in DPN results in DLss Aδ pain threshold energy more than twice as high as in age-matched controls. By contrast, C fiber pain thresholds were unchanged, and similar to those of controls.(10) This suggests that CMi fibers are relatively spared in PN, and that C-DLss selectively stimulates these sensitized C fibers to spontaneous activity. Overall, Diode Laser technology presents unique technical advantages compared to existing technology that makes it an ideal means to measure cutaneous axonal excitation, the basis for our proposed biomarker.

DLss has unique biomarker significance derived from its peripheral and neuropathic specificity. Why is a neuropathic pain surrogate Response marker useful and how might it aid in performance of clinical trials? Pain is an individualized, elastic, complex, summative and nearly ubiquitous human experience 75. Moreover, it is simple to measure with a wide array of patient-reported clinical instruments to record patient perception of severity of pain, its location, and dimensions that help to identify pain as neuropathic 76. However, the deeply subjective experience of pain poses challenges in quantitation, especially when measuring change in clinical trials 77. As recognized by HEAL and the NIH, pain perception benefits from correlative objective and scalable measures 5,41. C:Aδ ratio separates PN patients who experience neuropathic pain from those who do not, and Example 1 explain and confirm that DLss C:Aδ ratio correlates with participant's experience of peripheral neuropathic pain. Due to its specificity for neuropathic pain of peripheral origin, the DLss ratio biomarker is proposed primarily as a “Response” biomarker, defined by BEST, “A biomarker used to show that a biological response has occurred in an individual who has been exposed to a medical product or an environmental agent.” A change in DLss C:Aδ ratio during treatment likely reflect suppression of peripheral CMi fibers hyperexcitability by agents (Example 2), correlate with relief of pain, distinguish between active and placebo treatment arms, and qualify DLss as a BEST defined Response biomarker for these agents (Example 2).

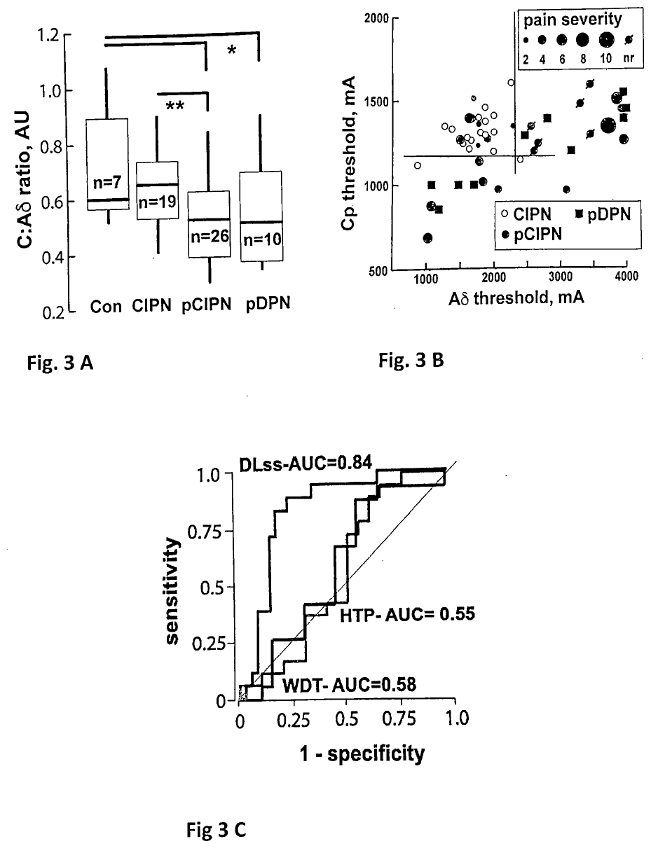

The development of a response biomarker specific to peripheral pain will have important implications.
Suppression of peripheral nociceptor spontaneous hyperactivity is ideal to achieve neuropathic pain symptomatic relief, and often more effective than centrally acting agents 78. A validated p-ONP biomarker would encourage the development of peripherally active agents. Lidocaine, which has a primarily peripheral mode of action in blockade of voltage gated Na+ channels, is a class prototype that is endorsed by the CDC 79 as an adjunctive agent for neuropathic pain suppression because of its efficacy, benign side effect profile, and resistance to tolerance. Lidocaine 5% patch is chosen as the proof-of-concept treatment in the proposed analytic validation Aims 2 and 3. Excitingly, several novel specific blockers for the voltage gated sodium channels Nav 1.7 and 1.8 have significant peripheral action 80, and DLss would assist in measuring pain response in similar agents. Effective peripherally acting agents would reduce the common but improper use of centrally acting opioids for ONP, and ameliorate the habituation, physical dependence and side effects of long term opioid use 77, 81, 82.
Quantitation of peripheral effect from agents with mixed central and peripheral effects. For novel agents similar to gabapentin, which have been shown to reduce pain mixed central and peripheral modes of action 83, use of DLss would help to define the contribution of peripheral action in early phase trials.
Amelioration of placebo effect as a confounder in clinical trials: Placebo response to perceived pain interventions is potent,(84) and has been recognized to cause negative results in pain double-blind studies (DBRCTs) 85. However, C:Aδ ratio was not significantly change despite improvement in pain experience after placebo treatment (Example 2). Therefore, change in C:Aδ ratio might be used to test the peripheral biological validity of perceived pain relief. It could be combined with patient-reported pain instruments in a dual primary endpoint to increase statistical power in future trials of peripherally- or mixed-acting neuropathic pain agents.
Appropriate selection of participants for clinical trials. This invention also demonstrated potential (Example 1) the DLss C/Adelta ratio biomarker utility as a BEST Diagnostic biomarker. It is likely that C:Aδ ratio can distinguish neuropathic pain from musculoskeletal pain, and peripheral neuropathic pain from that of CNS origin (e.g multiple sclerosis).
Further, C fiber pain threshold and Ad detection threshold, either combined as a ratio (FIG. 3a) or in a binary limits analysis (FIG. 3b) can distinguish painless from painful neuropathy. Eliminating painful PN patients who co-segregate with the DLss parameters of painless PN patients might improve statistical power of pain trials by excluding patients unlikely to respond to peripherally acting agents.

### Need for Practical Biomarker

There is no practical biomarker that significantly correlates to neuropathic pain of peripheral origin or monitors its effective treatment. C:Aδ ratio by DLss is a novel biomarker signature specific for peripheral neuropathic pain. Applying this biomarker in people with signs and symptoms of painful peripheral neuropathy could provide objective correlation to patient-reported pain outcome measures, qualifying as a BEST Response biomarker. Use of this biomarker may speed development and validation of novel pharmaceuticals with action specific to peripheral axonal pain generation in order to reduce progression from early acute to chronic neuropathic pain.

### Advantages of Diode Laser

Studies in animals, healthy subjects and patients find that DLss assessment of C or Aδ fibers is 1) a stable measure over time that 2) correlates to peripheral neuropathic pain of different origin, 3) provides assessment of key fibers (CMi) involved in mediation of ongoing, spontaneous neuropathic pain, 4) allows fiber selective and specific assessment efficacy of pain therapy, and 5) correlates to peripherally evoked pain with its representation in CNS.

Deep dermal CMi fibers are more easily stimulated when spontaneously active. Although time consuming and painful, microneurography, in which electrical activity of axons are recorded in skin or nerve bundles individually, allows identification, localization and classification of individual nociceptors and measurement of their activation state. Using microneurography to individually examine dermal and epidermal fibers 48,49, PI Nemenov showed that spontaneously active CMi fibers require a lower temperature of radiant heating for activation (40° C. skin warming sufficient to stimulate >50%, n=21) compared to CMi fibers that were not spontaneously active (45° C. required to stimulate 50%, n=53) 90. This supports the concept that CMi fibers may be selectively activated and are particularly susceptible when spontaneously active, as in painful PN.

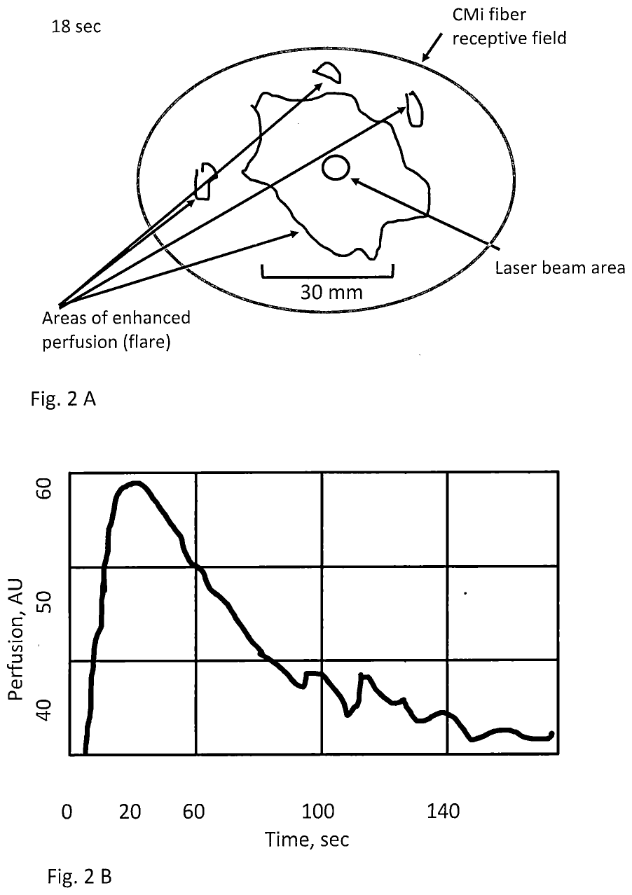

Axon Flare is a measurable manifestation of CMi fiber activation 15, 91 that may correlate with degree of peripheral neuropathic pain 92, 93. Axon flare describes the autonomic relaxation of cutaneous arterioles in the cutaneous distribution of an activated CMi fiber, resulting in increased cutaneous blood flow that can be captured using a Speckle Imager 94. Using axon flare as a specific measure of CMi activation, we examined if DLss stimulation could selectively evoke pain perception from CMi fibers without suprathreshold painful activation of other C polymodal fibers in healthy controls, and in pigs. CMi pain thresholds were defined by subject response. Neurogenic flare detection was correlated to CMi pain threshold in all 12 participants, flare area matched the receptive field of CMi fibers 95, and could be detected BEFORE pain perception in 10/12 (FIG. 2). No difference in mechanical sensitivity (mediated by polymodal C fibers) was observed between diode laser stimulated and surrounding skin suggesting DLss does not induced suprathreshold non-tolerable pain as traditional heat stimulators do. These studies reinforce the tolerability of DLss compared to other techniques and suggest that measurement of axon flare size and temporal profile may offer a sensitive correlate to patient pain perception as part of a composite response biomarker.

DLss is safe and reliable as a repeated measure found high reliability across repeated DLss for both component portions of the measure and for the resultant C:Aδ ratio. The area of skin stimulated by the diode laser is shifted with each trial to minimize heating.

C:Aδ ratio distinguishes between painful and painless neuropathy across neuropathy mechanisms Patients, ages 30-80, with neuropathy following neurotoxic chemotherapy with platinum agents or taxanes were examined with DLss and a battery of QST measures: warm and cold detection thresholds (WDT, CDT), heat and cold pain thresholds (HPT, CPT), mechanical pain threshold (MPT), and temporal summation to pinprick stimulation, and vibration. Participants rated their ONP severity using a 0-10 numerical rating scale. 26 CIPN patients (pCIPN) who reported pain of 2 out of 10 or more were compared to 19 CIPN patients who reported pain of 0 (CIPN). DLss and QST results from these patients were combined with data from a similar study of patients with painful diabetic neuropathy 10 as shown in FIG. 3. DLss C:Aδ ratio of detection thresholds were significantly different (p<0.05) between painful and painless CIPN patients by multivariant regression analysis accounting for age as a confounder. We anticipate DLss will distinguish painful from painless PN regardless of neuropathy origin. Graphical representation of C pain to Aδ detection thresholds in a scatter plot (FIG. 3b) demonstrates the physical separation between CIPN patients without pain in the upper left quadrant of the graph, and those with pain as well as patients with diabetic painful neuropathy, primarily arrayed outside this quadrant. This 2-dimensional separation may allow even more powerful segregation in a larger data set.

By contrast with DLss, QST measures (WDT, CDT, HPT, CPT, MPT, did not significantly distinguish between the painless and painful groups when age was taken into account. In receiver operator characteristic (ROC) analysis, HPT (FIG. 3c) and other QST measures performed much less well than DLss. Selected previous studies have reported that QST measures can distinguish painful from painless PN 14. However, HPT, and especially WDT outcomes are sensitive to age effects and may account for these findings. Overall, rigorous studies have not demonstrated that currently available QST measures, or any other biomarkers separate painful and painless PN and/or correlated to ongoing, spontaneous pain 14, 65, 96. DLss, if validated, would represent the first biomarker selective for pain of peripheral origin.

Among CIPN patients with neuropathic pain, there was a statistically significant correlation between reported pain severity and both DLss evoked C pain (Correlation coefficient=0.43), and C:Aδ ratio (CC=0.46). We will modify DLss to optimize correlation with neuropathic pain in Aim 1, and anticipate that the strength of this correlation will be further improved when controlling for age and neuropathy severity, and with the use of modified diode laser beam size to reduce variability of Aδ activation threshold. Overall, these results demonstrate the significant promise of DLss as a selective biomarker for peripheral origin of neuropathic pain.

DLss demonstrates selective decreased CMi excitability in response to lidocaine. Lidocaine is a wide spectrum sodium channel blocker that is used clinically as a dermal patch or intradermal injection 97-99 for treatment of peripheral neuropathic pain 3, 99. Lidocaine injection can completely block peripheral ONP 12. C and Aδ fibers are differentially affected by lidocaine treatment 99-101, and suppression of repetitive CMi activation that mimics spontaneous activity is a potent consequence of lidocaine treatment 100. Flare response in rhesus monkeys, a primate animal model for human neuropathic pain generation and nociceptor dynamics was similar to that observed in healthy subjects (FIG. 2). A total of four monkeys were tested. DLss induced flare was evaluated before and after intradermal 2.0% lidocaine injection and saline subdermal injection was used as a control. After the lidocaine injection axon flare area was markedly reduced compared to that seen after saline. In similar studies, DLss evoked similar flare response following topical application of capsaicin and carrageen 9,102,103. These studies reinforce the biological specificity of DLss for CMi activation, and demonstrate that DLss can serve as a biomarker for response to treatment.

### Example 1 Detail Description

DLss C:Aδ ratio as a specific correlative Response biomarker of peripheral neuropathic pain generation.

Participants: patients with diverse etiologies of sensory predominant peripheral neuropathy (N=50, Utah), and 10 controls without neuropathy or orthopedic foot pain (Stanford). For Aim 1, (and 2 and 3, see below), peripheral neuropathy patients, age 30-79 was recruited.

Eligibility Criteria

Neuropathy participants have a slowly progressive, length dependent, sensory predominant neuropathy as judged by the Investigator, meeting phenotypic Toronto Diabetic Neuropathy Expert Group consensus criteria for “probable sensorimotor peripheral neuropathy” with symptoms present for at least 3 months (including for those with CIPN). Expected etiologies include genetic, nutritional, drug and chemotherapy induced, diabetic, associated with prediabetic metabolic syndrome, and idiopathic. Patients with acute autoimmune (e.g. Guillain Barre Syndrome) or toxic neuropathies (e.g. arsenic, solvents) were excluded, as will patients who report significant co-existent musculoskeletal foot pain as well as severe edema, other skin damages.
Baseline Visit. To define baseline C and Adelta thresholds
100 mm Visual Analogue Scale (VAS), serves as the primary measure of neuropathic pain in response to the single question “Please report the average severity of your foot or distal leg pain over the past 12 hours, where the left edge of the scale represents no pain, and the right represents the worst pain imaginable”. Pain VAS is well validated in clinical trials106, and selected for this study in preference to an 11-point numeric rating scale (NRS) because it is shows less anchoring bias than NRS, important for subsequent lidocaine trials 107, 108. Participants will be given paper VAS forms to record foot-focused pain VAS twice daily during the 7-day interval between Screening and Baseline visits. A 6-hr window will be available for each recording, and results will be timestamped. Adequate compliance will be considered completion of 8 of the possible 14 timestamped VAS reports. Average reported VAS over the 7-day recording period will serve as the primary neuropathic pain determinant for Aim 1. The following measures will also be obtained to provide an anchoring comparison to the VAS.
Standardized PROM IS questionnaire comprising 3 pain severity and 6 pain interference questions 109.
Brief Pain Inventory. This pain inventory consists of 3 pain severity items and a 7-item pain interference scale specifically validated for PN 110.

### Description of Methods and Devices Used in Examples 1 and 2(A,B)

Diode laser for selective stimulation (DLss) testing of C:Aδ ratio. Detection and pain thresholds for fiber-selective laser stimulation was performed with Lass-10M, as previously described (patents U.S. Pat. No. 7,402,167B2 and U.S. Pat. No. 8,029,553B2). The foot dorsum was shaved to limit light absorption and overheating. Skin temperature on the dorsal foot will be monitored and maintained at 32-33° C. using periodic heating pad warming.
Laser detection and pain thresholds (separately for Aδ and C fiber stimulation) were quantified using the “method of levels” on a 6×6 cm stimulus grid drawn on the foot dorsum, shifting location and using a series of ascending stimulations starting from sub-detection intensity, increasing by fixed increments with the highest stimulation determined by participant reporting a painful sensation with intensity between 30 and 40 on the 0-100 VAS. C fiber stimulation will ramp from 800 to 1700 mA in 100 mA increments using a 5 mm beam. Aδ fiber stimulation intensity will ramp from 1000 to 4000 mA in 200 mA increments using a 1 mm stimulus diameter.
DLss axon flare is a cutaneous microvascular manifestation of selective activation of CMi fibers 15. Blood perfusion was recorded by Speckle Imaging (PeriCam PSI System, Perimed, Sweden), following DLss stimulus using the C fiber protocol above. Testing at sequential sites was performed at 4 min intervals to allow flare dissipation from previous trials.

C (pain thresholds): Aδ (detection thresholds) ratio was used as the primary correlative biomarker of patient reported p-ONP. The intensity and area of DLss induced flare perfusion will be evaluated to see if it adds correlative power to C:Aδ ratio.

Measures of Neuropathy Severity
UENS is a validated defined examination scale with a range of 0-42, specific for early sensory predominant peripheral neuropathy(105). A score >4 suggests neuropathy.
3 mm punch skin biopsy at the distal leg and distal thigh for IENFD, was performed in the same leg as Tissue will be stained with PGP-9.5, and IENFD determined by a technician, blinded to participant status, using current criteria counting dermal-epidermal crossing fibers only, and also including fragments. Distal thigh IENFD and UENS score will be used as a co-primary measures of neuropathy severity for statistical correlation analysis. The following validated measures are included because they record patient experience and large fiber specific that are complements to the primary measures.

### Control QST Measurement

Functional measurement of small fiber neuropathy. QST for warm detection (WDT) and heat pain thresholds (HPT): was be determined using a Thermal Sensory Analyzer (TSA-II, Medoc, Ramat Yishai, Israel) 111, 112, identical to that used in preliminary studies. The thermode with contact area of 9.0 cm2 will be applied to the foot dorsum, and thresholds determined by continuous ramping of temperature from 32° C. baseline temperature by 1° C./s until the subject presses a ‘stop’ button, with a maximum cutoff of 50° C. to avoid injury for HPT. The average threshold is calculated from three measurements in each area(12).

### Extended Outcome of Example 1

(Example 1) In PN participants DLss C:Aδ ratio correlate significantly with averaged pain VAS, p<0.01, regardless type and severity of PN. Repeated measure reproducibility for C:Aδ ratio exceed 85%. UENS correlated with IENFD, p<0.05 but we the DLss C:Aδ ratio biomarker was not significantly correlate with UENS or IENFD. This confirms that the DLss biomarker reflects severity of neuropathic pain, but not of neuropathy severity. Axon flare measurement yield higher correlation with reported average pain VAS than was C:Aδ ratio alone. The multivariant statistical analysis was performed.

### Extended Outcome of Example 2A

DLss as a Response biomarker for peripherally acting neuropathic pain agents.

Based on example 1 the change in the DLss C/Adelta ratio biomarker accurately reflect change in participant's report of neuropathic pain in response to a peripherally acting neuropathic pain agent. As a model of the class of peripherally active neuropathic pain agents for which the DLss biomarker will be most applicable, we examined response to lidocaine, applied to the foot dorsum as a 5% topical patch. Lidocaine acts specifically to block depolarization of Nav 1.7 and Nav 1.8 voltage gated Na+ channels on C and Aδ cutaneous nociceptors to reduce spontaneously generated afferent axonal signaling 113, 114. Although preclinical models suggest minor anti-inflammatory 113, and possible central effects of lidocaine 115, primary effects are peripheral, consistent with the specificity of the candidate biomarker. Commercial patch formulations deliver controlled release over 12-16 hours, are well tolerated, have minimal drug interactions with centrally acting agents. Cross-over design was used, because the pairwise between-treatment comparison offers greater statistical power.

Recruitment, screening and eligibility: 44 total PN participants, age 30-79, were recruited as described above. Eligibility for Example 2A is the same as Example 1, with the addition that participants must report sustained significant neuropathic pain, as validated by their report of average VAS >40 mm over the 7 day lead-in period. Baseline neuropathy and pain phenotyping consisting of pain self-report instruments, and UENS. They will undergo the full battery of Baseline QST and DLss measures as described above. Change in the DLss C:Aδ ratio measure over the treatment period was served as the Response biomarker.

Lidocaine patch cross-over trial: For ease of scheduling and to optimize compliance, study visits for Example 2 was set up on the same weekday 7 days apart. Subjects were randomized in a cross-over design to first apply either 5% lidocaine patches or placebo patches to the distal dorsum of the foot bilaterally. First application of the study patches will be completed at the Baseline visit. The participant, study staff, and Investigator will be blinded to treatment order. Patches worn for 12 hours each day for a total of 7 days. Change in average VAS between the 7-day post-screening period, and the final 4 measurements during the treatment periods (and washout period) served as the Primary Endpoint for efficacy.

Day 7 and 21 treatment evaluation visits, and washout days 7-14: After 7 days of study patch treatment, participants returned for evaluation. All Baseline pain including, the UENS, QST, and DLss will be repeated. A 7-day washout period will then ensue, with no patches, followed by a 7-day period of treatment with the other patch type, and finally repeated measures as above.

### Extended Outcome Example 2

During treatment with 5% lidocaine patch participants have significantly greater improvement in VAS than during the placebo patch phase, p<0.05. Change in DLss Cpain/Adelta-det ratio using pairwise comparison was significantly correlate with change in the reported VAS, p<0.01. These results demonstrated that the efficacy of DLss biomarker as a Response biomarker. UENS was not change significantly during treatment periods, and this stability serve as evidence that change in p-ONP or its biomarker does not reflect change in neuropathic distal axonal injury.

Extended Description of Example 2B

Extended Verification Efficacy of C/Adelta Response Biomarker)

More rigorous parallel treatment arm approach to compare 7-day response to either 5% lidocaine or placebo patch. All study procedures are the same as in Example 1
Recruitment of 80 participants use eligibility criteria, clinical venues and methods as described in Example 1.
Screening evaluation for PN, as described in Example 1 with reporting foot-specific neuropathic pain VAS more than 40 mm.
Baseline evaluation for this validation trial as for Example 1. Baseline QST and DLss measures as described in Example 1.
Lidocaine parallel arm, double blind, placebo controlled trial: the participants who met eligibility criteria were randomized to apply either 5% lidocaine patches or placebo patches to the distal dorsum of the foot bilaterally for a 7 day period, using the same education, double blinding, treatment protocol, monitoring twice daily VAS self-report, measures of compliance, potential dropout rates, and change in pain VAS Primary Endpoint measure as described in Example 2A.
Day 7 treatment evaluation visit: After 7 days of study patch treatment, participants will return for evaluation. All Baseline pain and mood assessments, the UENS, QST, and DLss will be repeated.

Outcome: Participants randomized to 5% lidocaine patch had significantly greater improvement in VAS than will those randomized to placebo patches and about 50% reduction in VAS severity, while placebo treatment reduced VAS by less than 20%. DLss ratio will significantly distinguish the lidocaine treatment group from the placebo group, p<0.01. Among those treated with lidocaine, DLss significantly correlate with VAS treatment response, P<0.01. These results confirm that the DLss biomarker can serve as a BEST-defined “Response” biomarker for validation of new pain therapy in clinical trials. Separately, application of a pre-specified alternate DLss/pain VAS combined Secondary Endpoint showed superior statistical power to distinguish between treatment groups compared to VAS alone.

Statistical Analysis

Appropriate statistical analysis was used including multivariable.

What is claimed is:

1. A process for utilizing a diode laser to illuminate at least one A-delta nerve fiber and at least one C nerve fiber of a patient to produce a mechanistic biomarker for measuring efficacy of a pain therapy, said process comprising steps of:
A) utilizing the diode laser to illuminate the alpha delta nerve fibers and the at least one C nerve fiber with a plurality of laser beams to produce a set of intensity (from detection to pain threshold) responses,
B) measuring the DL intensity that evoke responses of C and A-delta fiber(s),
C) creating set of ratios of the intensity responses,
D) applying a pain therapy to the patient,
E) repeating steps A), B), C) and D)
F) utilizing the ratios to determine the efficacy of the pain therapy.

2. The process of claim 1 wherein the patient is a human.

3. The process as in claim 1 wherein the diode laser is adapted to produce laser beams at wavelengths of at a desired wavelength between 600 nm and 2000 nm.

4. The process as in claim 1 wherein the diode laser is adapted to produce laser beams at wavelengths of at a desired wavelength of 980 nm.

5. The process of claim 1 wherein the patient is suffering from chronic pain.

6. The process as in claim 1 wherein the diode laser is adapted to produce laser beams with a diameter of between 0.5 mm and 2 mm for illuminating the at least one A-delta nerve and diameter of between 3 mm and 15 mm for illuminating the at least one C nerve fiber.

7. The process as in claim 1 wherein the diode laser is adapted to produce laser beams with a power of between 6 W and 30 W for illuminating the at least one A-delta nerve and a power of between 0.6 W and 6 W for illuminating the at least one C nerve fiber.

8. The process of claim 1 wherein the measured intensity response is an intensity of painful or non-painful sensation.

9. The process of claim 1 wherein the measured intensity response is a first recognition (detection threshold) of any sensation pain.

10. The process of claim 1 wherein the measured intensity response is an intensity of power to produce a first recognition of pain (pain threshold).

11. The process of claim 1 wherein the measured intensity to response is an intensity laser current to produce a specified level of sensation painless or painful

12. The process of claim 1 wherein the measured intensity response is an intensity of pain.

13. The process of claim 1 wherein the measured intensity response is an intensity of power to produce a specified level of pain.

14. The process of claim 1 wherein as a substitute for C fiber thresholds measurements the measurements of axon reflex flare described in patent application #US20190008440 is used

15. The process of claim 1 wherein as a substitute for A-delta fiber detection or pain thresholds diode laser intensity that evoke pain or detection thresholds of C fibers for DL with wavelength in the range of 1400 nm-1,900 nm is used

16. The process of claim 1 wherein the ratio changes could be used as a measurement of efficacy of particular pain medicine for particular patient and allowed to accelerate a process of finding of effective treatment from 6-12 weeks to 1-2 weeks.

17. The process of claim 1 wherein the ratio allows to predict efficacy of pain treatment select patient group that unlikely will be treated with developing by exclusion of pain patients group that is overlapped with non-painful group and healthy subjects by value of Ad and C thresholds and C:Ad ratio (example of realization FIG. 3B preliminary results)

18. The process of claim 1 wherein ratio is used as translatable biomarker from animal subjects to patients.
